(19) United States
(12) Patent Application Publication
(10) Pub. No.: US 2006/0235515 A1
(43) Pub. Date: Oct. 19, 2006

(54) Capsular tension ring, method for making a capsular tension ring and capsular ring and intraocular lens assembly

(76) Inventor: Christophe Chassain, Saint Clement De Riviere (FR)

Correspondence Address: YOUNG & THOMPSON, 745 SOUTH 23RD STREET, 2ND FLOOR, ARLINGTON, VA, 22202, US

(21) Appl. No.: 10/541,673

(22) PCT Filed: Dec. 30, 2003
(86) PCT No.: PCT/FR03/03942

§ 371 Date: May 3, 2006

(30) Foreign Application Priority Data
Jan. 8, 2003 (FR) 03/00143

(51) Int. Cl. A61F 2/16

(52) U.S. Cl. 623/6.16; 623/6.42

(57) ABSTRACT

A capsular tension ring implantable in the equatorial region of a capsular bag after cataract ablation includes an open or closed annular body, with sharp edges and an axial width ranging between about 0.3 and 0.6 mm, or preferably about 0.5 mm. The annular body of the capsular tension ring, including the sharp edges, is made of a rigid material for the major part of the circumference, and includes at least one joint made of flexible material between two segments of the annular body made of rigid material. The invention also concerns an assembly including a capsular tension ring and an intraocular implant of the type including a central optical part and a peripheral haptic part having one or several haptic elements extending radially from the optical part, the optical part having a peripheral edge with a sharp ridge on its rear surface.

The present invention relates to capsular tension rings that are designed to be implanted in the capsular sac after ablation of the crystalline lens affected by a cataract in association with an intraocular lens designed to replace the crystalline lens.

These rings, known as “capsular tension rings”, have been adopted to preserve the shape of the capsular sac and to limit its post-operative shrinkage.

More recently, capsular tension rings have been designed to combat the opacification of the posterior capsule of the capsular sac causing secondary cataracts, rendering the posterior capsule opaque and necessitating the opening thereof by means of a YAG laser beam to restore visual acuity.

The opacification of the posterior capsule has two aspects: firstly, there are fibroses corresponding to in situ metaplasia of the anterior epithelial cells. This is a complication that arises in 40% of cataract ablation procedures entailing replacement of the crystalline lens. It is found that fibroses have little impact on visual acuity. Secondly, there are also Elschnig beads that form from germinative cells remaining at the equator of the capsular sac after the surgical procedure. These cells migrate along the posterior capsule and form opaque beads that are the main cause of reduced visual acuity linked to opacification of the posterior capsule.

Tension rings are not normally effective as barriers to cellular migration because they often have no sharp edges and/or have axial or anterior-posterior dimensions that are too small, allowing insertion of zonular fibers over the capsule, thereby maintaining an open germinative equatorial region.

Anti-opacification capsular tension rings are also known in the art. Most commercially available anti-opacification capsular tension rings are made of polymethylmethacrylate (PMMA), which is a hard and rigid material enabling sharp edges to be obtained that form an effective barrier to cellular migration. Manipulation of such rings by surgeons is a serious problem because they are very rigid and very elastic, which makes them dangerous during insertion into and deployment within the capsular sac. This is because, if forceps are used, after being passed through a corneal or sclero-corneal incision, a first end is inserted through the rhexis into the capsular sac, subtending an angle of approximately 270°, after which the remainder is bent over within the rhexis before it is released.

If an injector is used, the ring is placed in a tube and moved by means of a sliding hook passing through an eyelet at one or both ends of the ring.

In both cases, when released, the section of the ring made of rigid material such as PMMA can impact violently on the equatorial region of the capsular sac with the risk of causing lesions of the sac or tearing of the zonules attached to the exterior of the capsular sac at the equator.

Moreover, these capsular tension rings must necessarily be of the open type because, if they were configured as closed rings, the corneal or sclero-corneal incision would have to be much too large to allow the fast healing expected by surgeons and patients.

Hara et al. in “Efficacy of Equator Rings in an Experimental Rabbit Study”, Arch. Ophthalmol, v. 113, August 1995, pp. 1060-1065, propose a closed contour anti-opacification ring made from a flexible material, in particular from silicone, with sharp edges intended to constitute a barrier to cellular migration. This ring can be introduced into the eye through a relatively small (4.5 mm) incision.

The reduced rigidity of flexible materials known in the art prevents their use for the fabrication of open contour capsular tension rings. Moreover, the sharp edge of a flexible material ring does not constitute as effective a barrier to migration as the sharp edge of a rigid material ring, precisely because of the flexibility of the material, which allows the epithelial cells to pass.

EP-A-0 884 031 describes a generally C-shaped open capsular tension ring with sharp edges for producing a “wedging” effect on retraction of the capsular sac which blocks the migration of epithelial cells. The capsular tension rings described in the above document have a large (0.7 mm) axial or anterior-posterior width and a width of 0.2 mm to cover the whole of the width of the equatorial region of the capsular sac, from which the cells propagate.

A capsular tension ring of the above kind having a large axial width opposes fibrosis and prevents the sticking or capsular symphysis that is essential for stopping the migration of epithelial cells, or even Elschnig beads, onto the posterior capsule, inside the region corresponding to the periphery of the optic of the implanted intraocular lens replacing the crystalline lens. Symphysis is also advantageous in that it stabilizes the intraocular lens positioned in the sac.

It is also found that germinative cells are inevitably present inside the capsular tension ring, following incomplete capsular cleaning, because of myosis. These cells pass readily behind the optic, even if it has a sharp edge at the rear intended to constitute a barrier to such migration. This is because this sharp edge is effective, failing capsular symphysis, only if there is intimate contact with the posterior capsule.

Moreover, at present there is no rigid material anti-opacification capsular tension ring made in one piece with an optic intended to replace the excised crystalline lens that can be introduced and implanted under acceptable conditions.

The subject matter of the invention is a capsular tension ring that provides a barrier to equatorial cellular migration toward the posterior capsule and alleviates the drawbacks of prior art anti-opacification capsular tension rings.

A first aspect of the invention provides a capsular tension ring that is adapted to be implanted in the equatorial region of a capsular sac after ablation of a cataract, comprises an open or closed annular body having sharp edges over the vast majority of the circumference and an axial length from about 0.3 mm to about 0.6 mm, preferably about 0.5 mm, and is characterized in that the annular body, including the sharp edges, is made from rigid material over the majority of its circumference and includes at least one flexible material junction between two segments of the rigid material annular body.

The flexible material junction(s) constitute(s) a preferential bending region. In the conventional method of inserting an open capsular tension ring using forceps, a first end, referred to as the front end, of the ring is introduced and the ring is pushed in, turning it in the sac, until approximately three quarters of the annular body have been introduced. The annular body is divided by the flexible material junction(s) into two or more rigid segments allowing a gesture that is easier to control through avoiding movements of large amplitude liable to produce lesions. In practice, the sharp edges of the segments extend over at least 90% of the circumference of the annular body.

When a capsular tension ring of the above kind that has an open contour is easier to introduce into the capsular sac with the aid of an injector, these flexible material junctions provide increased flexibility enabling the ring to adapt better to the configuration of the tube, which typically is either rectilinear or has a very large radius of curvature relative to the radius of the capsular tension ring; this is achieved without risk of the annular body cracking, each of the rigid material segments of the implant being relatively weakly loaded.

The first end, or front end, of the open contour capsular tension ring, i.e. that by which the ring will be inserted, preferably comprises a flexible material and slightly re-entrant terminal portion with no eyelet that reduces or even eliminates the risk of perforating the sac. During post-operative shrinking of the capsular sac, the forces of which are damped by deformation of the ring, it is the other (second or rear) end that is preferably provided with an eyelet lug for manipulating the ring in situ, which can fit inside this flexible material terminal portion, minimizing the circumferential discontinuity.

If there is one junction, it is preferably approximately 300° from the front end.

In another embodiment, there are three junctions at approximately 120° to each other.

A ring of the above kind is more particularly intended for implants in which the optic has at least one sharp edge and two or more haptic elements adapted to bear on the main portion of the rigid segments of the capsular ring, achieving both good stability and good centering. The periphery of the optical portion provides a second effective barrier against cellular migration thanks to symphysis of the anterior and posterior capsules, despite the capsular tension ring having an axial width that is sufficient to block the proliferation of epithelial cells in the equatorial region.

A closed capsular tension ring preferably has two flexible material junctions, enabling marked ovalization or flattening of the implant and therefore introduction of the ovalized or flattened ring through a corneal or sclero-corneal incision much smaller than that needed for a rigid material closed contour ring. This kind of capsular tension ring with most of its sharp edges consisting of a rigid or hard material is a more effective barrier to cellular migration than a flexible material closed contour capsular ring.

Given that in practice the flexible material junctions are small (having dimensions of approximately 0.4 mm to 0.5 mm), conventional fabrication methods by assembling, bonding, fusing or overmolding rigid material segments and flexible material junctions would be difficult if not impossible to use. This is why fabrication by selective chemical modification of rigid material segments of an annular body that is made entirely of flexible material at the outset or of junctions of an annular body that is made entirely of rigid material at the outset, using the technology disclosed in the application EP-1003446, is to say the least highly advantageous. It goes without saying that, in the eventuality of there being one flexible material end, the latter will be obtained in the same manner as the junctions.

The invention and its advantages will be better understood in the light of the following description of preferred embodiments of a capsular tension ring according to the invention, which description is given by way of example and with reference to the appended drawings, in which:

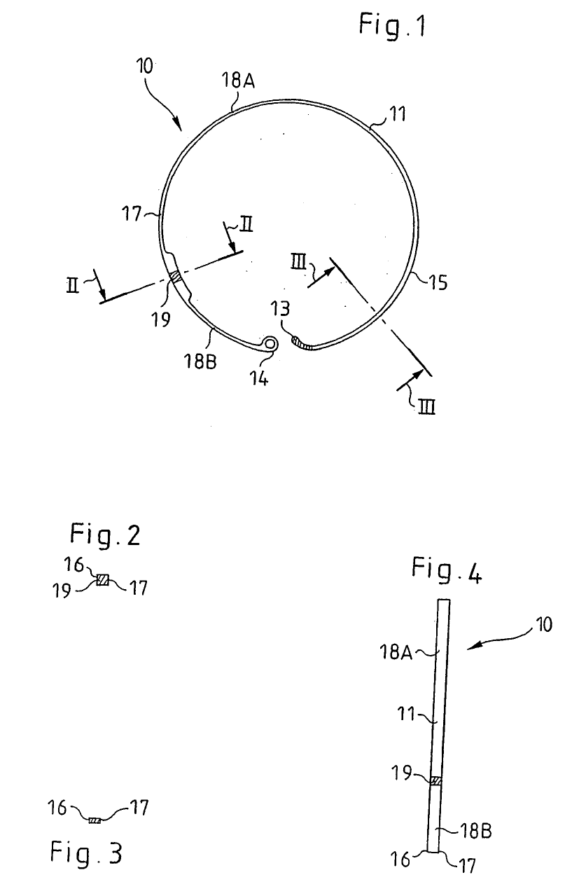

FIG. 1 represents a first embodiment of an open contour capsular tension ring of the invention;

FIGS. 2 and 3 are views in section taken along the lines II-II and III-III in FIG. 1, respectively;

FIG. 4 is a view in elevation of the capsular tension ring from FIGS. 1 to 3;

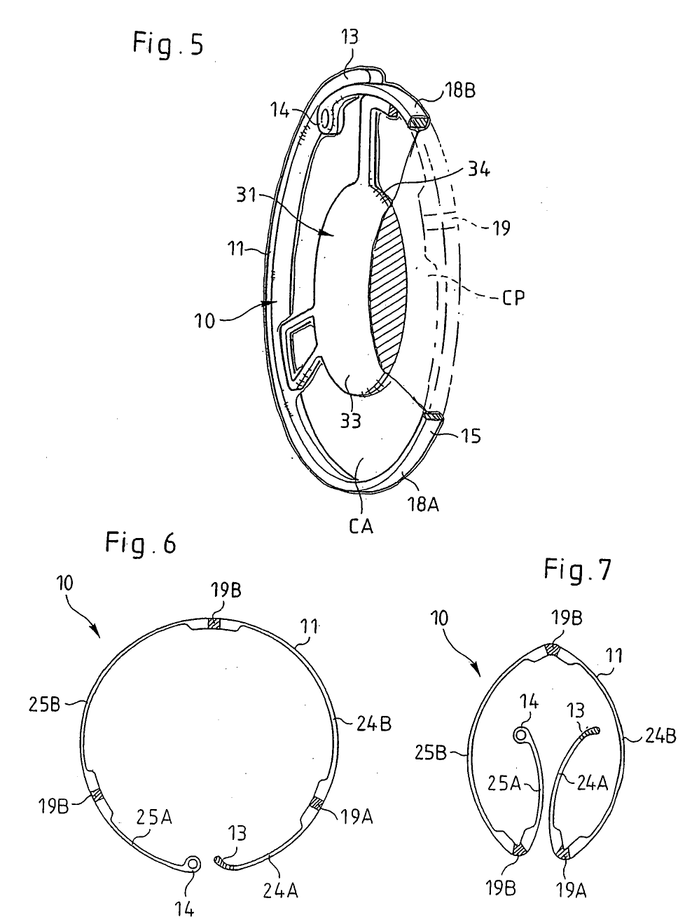

FIG. 5 represents the first embodiment implanted in the capsular sac after shrinkage of the sac and symphysis of the anterior and posterior capsules;

FIG. 6 represents a second embodiment of an open contour capsular tension ring of the invention;

FIG. 7 represents a bending mode of the first embodiment;

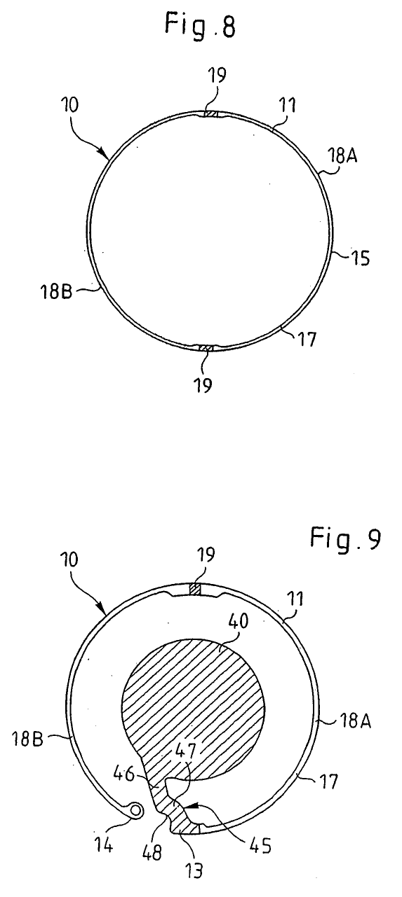

FIG. 8 represents a third embodiment of a closed contour capsular tension ring of the invention; and

FIG. 9 represents a one-piece assembly comprising an open contour capsular tension ring and an intraocular lens.

The capsular tension ring 10 represented in FIGS. 1 to 5 comprises an open contour substantially circular annular body 11. The diameter of the ring in the relaxed state is greater than that of the capsular sac into which it is to be implanted, in practice from about 10.5 mm to about 11.5 mm. The main portion of the annular body 11 has a first end 13 and a second end 14. In the relaxed state, the ends are circumferentially spaced from each other by about 0.5 mm. The annular body has a substantially rectangular radial section whose axial dimension is about 0.5 mm and is greater than its radial dimension, which is from 0.1 mm to 0.3 mm and preferably about 0.15 mm (see FIG. 3). The exterior surface 15 of the annular body is preferably substantially a right circular cylinder extending between two sharp edges 16 and 17.

The first end 13 of the capsular tension ring is in practice the front end that is introduced into the anterior capsule of the capsular sac via a sclero-corneal incision and the rhexis and is made from a flexible material so that the risk of accidental perforation is reduced in the event of a contact of its free edge with the tissues of the capsular sac. Furthermore, its flexibility facilitates its passage over the equatorial region of the capsular sac when the ring is pushed progressively along the equatorial region of the capsular sac, in the circumferential direction. This first end is slightly curved and re-entrant with the rounded free edge thicker than the main portion of the body of the ring. Thanks to this configuration of the flexible material first end, there is no risk of snagging or of perforation when the ring is introduced progressively into the capsular sac and no risk of accidental perforation of the capsular tissue on releasing one or the other segment or on turning the ring as it is introduced progressively into the capsular sac.

The second end 14 comprises a lug of a type known in the art that includes an eyelet and is offset inward and adapted to receive the hook of a manipulation instrument for modifying the position of the ring in the capsular sac or to be attached to a capsular tension ring injector rod, also known in the art.

The greater portion of the annular body 11 consists of a rigid or hard material which is weakly hydrophilic or weakly hydrophobic, in particular PMMA. In this embodiment, the annular body 11 comprises two annular segments 18A, 18B, the first annular segment 18A preferably subtending an angle greater than about 260°, less than about 320° and preferably equal to about 300° and the second annular segment 18B subtending an angle of about 55°.

A junction or block 19 of flexible material, such as hydrophobic or hydrophilic acrylic, of the type used for intraocular implants in particular, is localized between the two segments 18A and 18B. The junction or block 19 has a rectangular section, preferably a square section, with an exterior surface that preferably is substantially the shape of a right circular cylinder extending between two sharp edges continuous with the annular sharp edges 16, 17 of the segments. The substantially square section of the junction 19 is larger than that of the main portion of the annular segments, and therefore larger than that of the annular body, and has an axial dimension of about 0.4 mm to about 0.5 mm and a radial dimension of about 0.4 mm to about 0.5 mm, the portions of the segments 18A, 18B adjacent the junction having the same cross section as the latter. The circumferential extent of each of the junctions is from 0.5% to 6% of the circumference of the ring. For reasons connected with fabrication and mechanical strength, the opposite rims of the portions adjacent the junction converge radially toward the interior of the annular body. In any event, the exterior surface of the annular body is preferably substantially the shape of a right circular cylinder from one end to the other, whether it be the flexible material junction(s) or the rigid material segments, including the portions adjacent the junctions.

In the present embodiment, and in all the embodiments described herein, the capsular tension ring is in practice fabricated by selective chemical structural modification by the process disclosed in EP 1003446 of an annular body whose geometry corresponds to that of the final annular body, in particular by molding or by machining from a blank or semifinished lens as in the fabrication of intraocular lenses. The initial annular body is preferably made from flexible material and the hardness or rigidity of the regions corresponding to the desired segments is obtained afterwards by selective chemical structural modification of the corresponding portions of the initial annular body. The flexible material portions of the annular body, i.e. the junction(s) and the first ends, are preferably obtained from statistical methacrylate copolymers of methylmethacrylate and hydroxyethyl-methacrylate (MMA-HEMA) cross-linked by adding a multifunctional agent such as diethyleneglycol dimethacrylate. The rigid material of the lens is preferably based on PMMA. Alternatively, the initial annular body is made from rigid material and the regions corresponding to the required junctions are obtained by selective chemical structural modification. In both cases covalent bonds are obtained between the flexible material and the rigid material of the annular body. Thanks to this fabrication process, the structural integrity of the ring is much greater than could be obtained with conventional methods. It is therefore possible to bend the rigid material segments 18A, 18B about the junction 19, both in the general plane of the annular body and in a plane oblique or transverse to the general plane of the annular body, in order to insert the capsular tension ring, without risk of cracking or of separation between the flexible material junctions and the hard or rigid material segments.

The flexible material constituting the junction(s) may have a glass transition temperature of about 35° C., with the result that the ring can have a folded configuration facilitating its insertion through the incision and the rhexis and another specific deployed configuration corresponding to that of the ring when implanted in the capsular sac. For the same purposes, and with the same functions, the flexible material may be a shape memory material.

For implantation with surgical forceps, the first segment 18A is pushed around the equatorial region until the junction 19 reaches the rhexis, after which the second segment 18B is folded inside the capsular sac in a plane oblique or transverse to the general plane of the first segment 18A before it is released, thereby enabling the relatively short second segment 18B to take up its place in the equatorial region without risk of lesion of the sac or of tearing of the zonules, thanks to the reduced size of the second segment and the damping effect of the flexible material junction, which reduces the impact of the segment with the tissue of the capsular sac.

The diameter of the capsular tension ring is selected so that, once implanted, it is slightly compressed against the equatorial region of the capsular sac. This compression has the effect of closing the capsular tension ring by moving its ends toward each other, the first end 13 passing outside the second end 14 and thus forming a very small step at the overlap of the ends 13, 14, as shown in FIG. 5. The resulting discontinuity is minimized by the small thickness and the inherent flexibility of the first end 13, which tends to be crushed radially between the end 14 and the capsular tissue at the level of the equator. Afterwards, in the post-operative period, the capsular sac tends to shrink, by about 0.5 mm to 1.5 mm in diameter, the consequence of which is to increase the overlapping length.

The above kind of capsular tension ring may also be implanted using an injector known in the art. This kind of injector has a substantially rectilinear housing, or possibly a curved housing with a very large radius compared to that of the capsular tension ring. Thanks to the junction 19, the stresses induced by the substantially rectilinear deployment or slightly curved deployment of the ring are greatly reduced, thereby minimizing the risk of cracking, in particular when the capsular tension ring is loaded into the injector and shipped in sterile packaging intended to be opened at the time of use, i.e. months after being packaged.

After implanting the capsular tension ring, the surgeon positions the intraocular lens inside the ring. The haptic elements are C-shaped, J-shaped or flat, with or without an aperture, and there are two or three of them, for example. Each is in contact with or bears against the cylindrical interior surface of the main portion of the annular body. In the example shown in FIG. 5, an intraocular lens 31 of the type described in the patent FR-A-2,745,711 has three haptic elements 32 with a large aperture extending from the periphery of the optic 31 and forms an assembly with the first embodiment of the capsular tension ring. The ring therefore advantageously serves also to center and locate the intraocular lens in the capsular sac. The axial width of the interior surface, which is about 0.5 mm in the preferred embodiment, provides a good bearing surface for the haptic elements of the intraocular implant.

In addition to post-operative shrinkage, the anterior and posterior capsules or sheets move toward each other. Given that the axial width of the capsular tension ring is preferably limited to about 0.5 mm, i.e. from 0.45 mm to 0.55 mm, it does not prevent symphysis of the capsules, more particularly at the periphery of the optical portion of the intraocular lens. As shown in FIG. 5, the optical portion 33 of the intraocular lens 31 has, on the posterior surface, at least one sharp edge 34 that penetrates slightly into a fold of the tissue of the posterior capsule, thereby forming a second barrier to the migration of epithelial cells or Elschnig beads and preventing opacification of the posterior capsule behind the optical element and the formation of a secondary cataract.

The FIG. 6 embodiment of the capsular tension ring differs from the embodiment of FIGS. 1 to 5 only in the presence of three junctions. The annular body includes a junction 19B diametrally opposite the ends 13, 14 of the open annular body, with two other junctions 19A, 19C about 120° from the junction 19B. In other words, the plurality of junctions is regularly spaced over the circumference of the open ring. The junctions 19A and 19C are therefore at about 60° from the respective ends 13, 14. The main portion and the flexible material junctions have the same radial section.

The method of implanting this kind of ring may be the same as that used for the first embodiment. However, another implantation method is feasible in which the two relatively short segments 24A, 25A are first folded, preferably inwards, but possibly outwards, relative to the two relatively long segments 24B, 25B, and these segments are moved toward each other or pivoted about the junction 19B. The short segments 24A, 25B may simply be moved toward each other, but it is preferable for one pair of segments 25A, 25B to be slightly offset (in the axial direction) behind the other pair of segments 24A, 24B, in order to minimize the overall size of the segments 24A and 24B farther to the right of the segments 25B, 25A (not shown), thanks to the flexibility of the central junction 19B. The four segments are therefore introduced simultaneously through the incision and then penetrate into the capsular sac via the rhexis. The two short segments 24A, 25A are preferably released first, either successively or simultaneously, after which the two long segments 24B, 25B bent around the junction 19B are released. Thanks to a plurality of junctions, the surgeon may use different manipulations and sequences to introduce the segments into the capsular sac and deploy them there. Furthermore, there remains the possibility of introduction by means of an injector.

It will be clear that having three or more junctions further reduces the stresses to which the capsular tension ring is subjected, in particular when it must adopt a substantially rectilinear configuration to be housed in an injector and stored in that position for weeks or even months.

In the embodiment shown in FIGS. 7 and 8, the capsular tension ring 10 has a closed contour. To this end, it preferably comprises two diametrally opposed flexible material junctions 19. The configuration of the rigid material segments 18A, 18B and the flexible material junctions 19 is as described with reference to FIGS. 1 to 5, with the exception of the extent of the segments and the elimination of the ends.

A ring of the above kind has the advantage of maintaining its diameter despite shrinkage of the capsular sac. Thanks to the sharp edges along its rigid material segments, it provides an excellent barrier to cellular migration, given the perfect continuity of the two sharp edges over the whole of the circumference of the ring.

A closed contour ring of the above kind can sometimes be introduced through a relatively small incision, of the order of 3.5 mm, although the incision has to be larger than that necessary for introducing an open contour capsular tension ring, which is of the order of 2.5 mm, thanks to the two flexible material junctions 19 that allow ovalization or flattening of the ring with a major axis passing through the junctions clamped between the jaws of forceps. The ovalized or flattened ring is then introduced into the capsular sac by passing one of its junctions through the corneal or sclero-corneal incision and then the rhexis; continuing to push on the other junction, the portion of the ring inside the sac opens and espouses the shape of the equator. It is then necessary to push the rear junction farther so as to make it enter the circle of the rhexis, before releasing it to assume its place in the capsular sac. In this embodiment, the junctions also serve as dampers to absorb a portion of the energy released when the ring relaxes in the capsular sac.

Implanting the ring is followed by implanting the intraocular lens in accordance with the standard practice using forceps or an injector. The haptic elements of the intraocular lens are in contact with or bear against the annular interior surface of the main portion of the ring, i.e. are offset relative to the junctions and the adjacent portions of the segments.

In a further embodiment, represented in FIG. 9, flexible material optics 40 are formed in one piece with the capsular ring 10. In the relaxed position of this capsular tension ring/intraocular lens assembly, the optic is slightly off-center relative to the axis of the capsular tension ring.

Instead of a first end forming a terminal portion, it constitutes a connection 45 with the optic 40 whose periphery has sharp edges. The connection 45 includes a straight, preferably neither radial nor tangential, first haptic portion 46 subtending an oblique angle at the periphery of the optic 40, followed by a curved second haptic portion 47 with a step 48 opening circumferentially toward the second end of the ring before rejoining the interior rim of the first end 13 of the ring 10. The step 48 of complementary shape is adapted to receive the second end of the eyelet lug when the two ends move toward each other, following post-operative capsular shrinkage. It is clear that this kind of assembly can equally be obtained by selective chemical modification. The implantation of this assembly comprising a capsular tension ring and an optic is also facilitated by the junction 19 between the segments 18A, 18B, and begins with the introduction of the second end 14 of the ring, rather that its first end 13; once the capsular tension ring is largely in place in the equatorial region, the folded optic and the haptic portion are released, enabling the optic to find its position and to be held centered relative to the capsular tension ring implanted in the equatorial region and thereby centered relative to the capsular sac.

All embodiments of the capsular tension ring may be impregnated beforehand with an anti-proliferation product and in particular with 5-FU. The method of impregnation and deployment after implantation of the ring preferably conforms to the teachings of the patent application WO 98/25652.

It goes without saying that the present invention is not limited to the embodiments described or to the preferred materials, but to the contrary encompasses all variants of structures, configurations and materials that are compatible with the subject matter of the present invention.

1-26. (canceled)

27. Capsular tension ring adapted to be implanted in the equatorial region of a capsular bag after ablation of a cataractous crystalline lens, the capsular tension ring comprising an open or closed annular body having sharp edges and an axial length from about 0.3 mm to about 0.6 mm, the annular body including the sharp edges being made from rigid material over the majority of its circumference and includes at least one flexible material junction between two segments of the rigid material annular body.

28. Ring according to claim 27, wherein the axial width of the annular body is about 0.5 mm.

29. Ring according to claim 27, wherein the axial width of the annular body is from 0.45 mm to 0.55 mm.

30. Ring according to claim 27, wherein the annular body is closed and includes at least two diametrally opposed junctions.

31. Ring according to claim 27, wherein the circumferential extent of the at least each of one junction is from about 0.5% to about 6% of the circumference of the ring.

32. Ring according to claim 27, wherein it is open and the at least one junction is from about 260° to about 320° from a first end.

33. Ring according to claim 32, wherein the open ring has a plurality of said junctions regularly spaced over its circumference.

34. Ring according to claim 33, wherein the open ring has three said junctions at substantially 120° to each other and four segments, two of which subtend an angle of about 60° and two of which subtend an angle of about 120°.

35. Ring according to claim 27, wherein the radial width of the at least one junction and the ends of the segments adjacent said at least one junction is greater than the radial width of the segments of the annular body.

36. Ring according to claim 33, wherein at least one of the ends of the annular body has a flexible material terminal portion that is re-entrant and has a rounded edge.

37. Ring according to claim 33, wherein one of the ends includes an eyelet.

38. Ring according to claim 33, wherein the radial width of the annular body outside the portions of the segments or segments adjacent at least one junction is from 0.10 mm to 0.3 mm.

39. Ring according to claim 27, wherein a plurality of junctions subtending each of said junctions is substantially the same angle.

40. Ring according to claim 27, wherein the rigid material is selected from the group consisting of PMMA and acrylic and the flexible material is selected from the group consisting of HEMA, hydrophilic acrylics and hydrophobic acrylics.

41. Ring according to claim 27, wherein there are covalent bonds between the flexible material and the rigid material of the annular body.

42. Ring according to claim 27, wherein the flexible material comprises cross-linked statistical methacrylate copolymers of methylmethacrylate and hydroxyethylmethacrylate (MMA-HEMA) and the rigid material is PMMA based copolymer.

43. Ring according to claim 27, wherein the rigid material constitutes a chemical modification of the flexible material or the flexible material constitutes a chemical modification of the rigid material.

44. Ring according to claim 27, wherein the flexible material constituting the at least one junction has a glass transition temperature of about 35° C.

45. Ring according to claim 27, wherein the flexible material comprising the at least one junction is a shape memory material.

46. Assembly comprising a capsular tension ring according claim 27 adapted to be implanted in the equatorial region of a capsular bag after ablation of a cataractous crystalline lens, the capsular tension ring comprising an open or closed annular body having sharp edges and an axial length from about 0.3 mm to about 0.6 mm, the annular body including the sharp edges being made from rigid material over the majority of its circumference and includes at least one flexible material junction between two segments of the rigid material annular body, and an intraocular implant of the type having a central optical portion and a peripheral haptic portion including one or more haptic elements extending radially from the optical portion, the optic portion having a sharp peripheral edge on its posterior side.

47. Assembly according to claim 46, wherein the intraocular lens and the capsular tension ring are in one piece and the optic and haptic portions of the intraocular lens and the connection between the haptic portion and a first end of the ring are made of flexible material.

48. Assembly according to claim 46, wherein the haptic portion has a step facing the second end to receive it when the ring is compressed.

49. Method of fabricating an implantable capsular tension ring adapted to be implanted in the equatorial region of a capsular bag after ablation of a cataractous crystalline lens, the capsular tension ring comprising an open or closed annular body having sharp edges and an axial length from about 0.3 mm to about 0.6 mm, the annular body including the sharp edges being made from rigid material over the majority of its circumference and includes at least one flexible material junction between two segments of the rigid material annular body, the method comprising the steps of preparing the flexible material annular body and then chemically modifying segments of the annular body to form rigid segments, junctions between the rigid material segments remaining of flexible material.

50. Fabrication method according to claim 49, wherein the ends of the rings remains of flexible material.

51. Method of fabricating an implantable capsular tension ring adapted to be implanted in the equatorial region of a capsular bag after ablation of a cataractous crystalline lens, the capsular tension ring comprising an open or closed annular body having sharp edges and an axial length from about 0.3 mm to about 0.6 mm, the annular body including the sharp edges being made from rigid material over the majority of its circumference and includes at least one flexible material junction between two segments of the rigid material annular body, the method comprising preparing the rigid material annular body and one or more regions of the annular body to constitute one or more flexible material junctions between rigid material segments.

52. Fabrication method according to claim 51, wherein the chemically modified regions is one of the ends of the annular body.
